(19) United States
(12) Patent Application Publication
(10) Pub. No.: US 2017/0295893 A1
(43) Pub. Date: Oct. 19, 2017

(54) FASHIONABLE PIECE OF JEWELRY THAT PROVIDES AROMATHERAPY

(71) Applicant: Coreena D'Alessandro, Redding, CT (US)

(72) Inventor: Coreena D'Alessandro, Redding, CT (US)

(21) Appl. No.: 14/976,528

(22) Filed: Dec. 21, 2015

Related U.S. Application Data
(60) Provisional application No. 62/094,359, filed on Dec. 19, 2014.

(51) Int. Cl. A44C 15/00; A44C 25/00; A61L 9/12

(52) U.S. Cl. CPC A44C 15/002; A61L 9/12; A44C 25/002; A44C 15/005; A61L 2209/133; A61L 2209/15

(57) ABSTRACT

The present invention is for an apparatus for use as a fashionable jewelry that includes a scent tube positioned in a top cover. The scent tube has a recess and at least one hole that extends from the recess to an outer surface of the scent tube. A replaceable stick insert is positioned within recess of the scent tube and includes a substance that provides one or more aromatherapy benefits. A rotatable bottom cover is movable between an open position and a closed position

### BACKGROUND OF THE INVENTION

### 1. Field of the Invention

The present invention relates to a piece of fashionable jewelry, and more particularly, a necklace having a replaceable element that conveniently provides aromatherapy to the user during use.

### 2. Description of Related Art

Aromatherapy is a form of alternative medicine that are known to alter a user's mood through the use of one or more of substances including, but not limited to, various plant materials, aromatic plant oils, including essential oils, and other aromatic compounds. The practice of aromatherapy includes providing a blend of the above materials to user through topical application, massage, inhalation or water immersion to stimulate a desired response.

Various devices are known in the art for delivering essential oils to the user. In some instances, the use of fashionable jewelry has been utilized. For example, U.S. Pat. No. 8,082,754 discloses a piece of jewelry to be worn by a wearer, having a spray mechanism mounted on the piece of jewelry, and a reservoir for containing a fluid mounted on the piece of jewelry, to dispense a fluid. The manipulation of the reservoir by the wearer actuates the spray mechanism and dispenses fluid from the reservoir.

Additionally, U.S. Pat. No. 4,964,570 discloses an article of jewelry for dispensing perfume consisting of a pair of hollow tubes one of which is provided with a sealed capsule of perfume and the other tube containing venting openings and a needle to penetrate the capsule when the two tubes are assembled and the needle is pushed far enough to make the penetration.

The prior art has shortcomings. Both of the above-cited prior art documents acts require the user to activate the device to achieve the desired effect. The present invention overcomes these, and other, shortcomings in the art.

### OBJECTS AND SUMMARY OF THE INVENTION

The present invention is an apparatus for use as a piece of fashionable jewelry that includes a scent tube positioned in a top cover. The scent tube has a recess and at least one hole that extends from the recess to an outer surface of the scent tube. A replaceable stick insert is positioned within recess of the scent tube. The replaceable stick includes a scent stick that includes a substance that provides one or more aromatherapy benefits. A bottom cover that is rotatable relative to the scent tube from an open position to a closed position.

A first object of the present invention is to provide piece of jewelry that is fashionable and also provides the user with one or more aromatherapy benefits.

Another object of the present invention is to provide a piece of jewelry that does not require the user to actively emit the substance that provides an aromatherapy benefit from the apparatus.

A further object of the present invention is to provide a piece of jewelry that is user adjustable so the end user can determine how much of the substance that provides an aromatherapy will be emitted from the apparatus, if any will be emitted at all.

An even further object of the present invention is to provide a piece of jewelry that can be worn many times along with a replaceable scent stick insert that, when worn out, can be replaced by the user so that the same, or different, aromatherapy benefits can be achieved time after time.

The above objects are achieved in accordance with the present invention.

### BRIEF DESCRIPTION OF THE DRAWINGS

The present invention will be more fully appreciated as the same becomes better understood from the following detailed description of the present invention when viewed in conjunction with the accompanying drawings, in which:

FIG. 1 is a front view of the apparatus of the present invention;

FIG. 2 is a cross-sectional view of the apparatus of FIG. 1 along line 2-2;

FIG. 3 is an exploded view of the apparatus of FIG. 1;

FIG. 4 is a front view of a top assembly with a replaceable scent stick insert therein with a bottom cover removed;

FIG. 5 is a front view of a removable scent stick insert;

FIG. 6 is a second embodiment of the apparatus shown in FIG. 1 shown with bottom cover rotated relative to the top assembly such that the vents are at least partially exposed; and

FIG. 7 is an embodiment of the apparatus shown in FIG. 1 shown with bottom cover rotated relative to the top assembly such that the vents are at fully covered.

### DETAILED DESCRIPTION

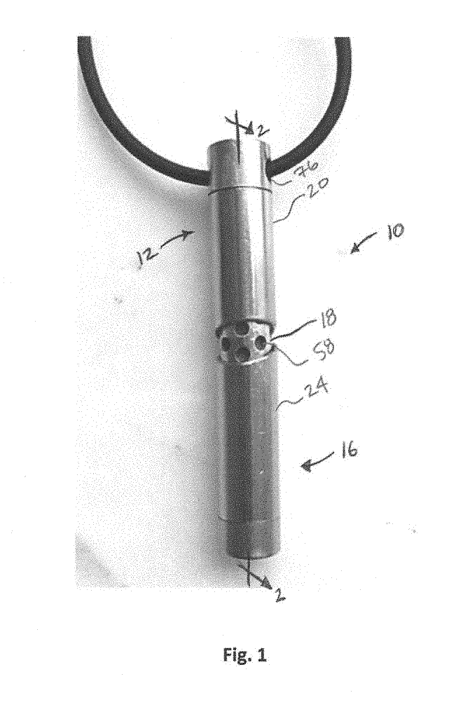

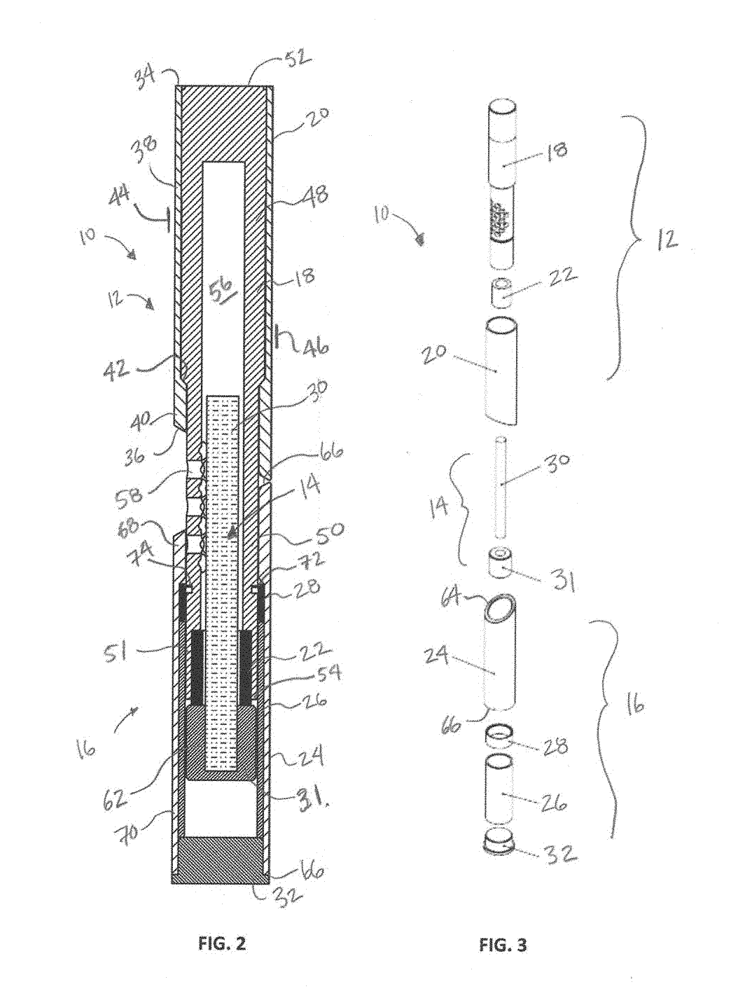

Referring to FIGS. 1 and 2, the apparatus 10 is a piece of fashionable jewelry that provides aromatherapy to the user that includes a top assembly 12, a replaceable scent stick insert 14, and a bottom assembly 16. The top assembly 12 includes a scent tube 18 that is inserted into a top cover 20. The bottom assembly 16 includes a magnet 22, a bottom cover 24, a spacer 26, a retaining clip 28, and a bottom insert 32. The replaceable aromatherapy stick insert 14 includes a scent stick 30 and a cartridge base 31.

Referring now to FIGS. 1-3, the top cover 20 is shown to be a hollow cylinder having an upper end 34 and a lower end 36. In addition, the top cover has an upper portion 38 and a lower portion 40. Although the outer and inner shape of the top cover 20 is shown to be round, the outer shape of the top cover 20 can be any shape desired by the designer. For example, although not shown, the outer shape of the top cover 20 can be triangular, square, hexagonal, etc. In some embodiments, the outer shape of the top cover 20 might vary from the upper end 34 to the lower end 36. The inner shape of the top cover 20 can also be any shape desired by the designer; however, a round shape is preferable to enable the top cover 20 to be conveniently rotated relative to other aspects of the apparatus 10 for added functionality described infra. As shown in FIG. 2, preferably, the diameter of the inner shape of the upper portion 38 of the top cover 20 is slightly larger than the diameter of the lower portion 40 of the top cover 20. The slight difference in diameter size between the upper and lower portions conveniently creates a ledge 42 that is useful when positioning the scent tube 18 in the top cover 20. In the embodiment shown, the ledge 42 provides a slight angled surface. As is clearly shown in, e.g., FIGS. 2 and 3, the upper end 34 of the top cover 20 is generally at a 90 degree angle relative to the length of the cylindrical shape. The lower end 36 of the top cover 20 is preferably angled at an angle less than 90 degrees such that the length of the top cover 20 on a front side 44 is shorter than the length of the top cover at a rear side 46. The top cover 20 can be made of any material; however, plastics and metals are preferable. Most preferably, the top cover 20 is made of brass.

Referring now to FIGS. 2 and 3, the scent tube 18 is generally a cylinder having a top portion 48, a middle portion 50, and a bottom portion 51. The top portion 48 has a diameter that is sized to slide into the upper portion 38 of the top cover 20. The middle portion 50 and bottom portion 51 have outer diameters that are slightly smaller than the top portion 48. Together the middle and bottom portions 50, 51 are sized to slide through the lower portion 40 of the top cover 20. The top portion 48 of the scent tube 18 preferably has a length that, when inserted into the top cover 20, extends from the ledge 42 to upper end 34 of top cover 20 (see e.g., FIG. 2) such that the top end 52 of the scent tube 18 is generally flush with the upper end 34 of the top cover 20. The middle portion 50 and bottom portion 51 of the scent tube 18, together, have a length that extends through the lower portion 40 of the top cover 20 such that the bottom end 54 of the scent tube 18 is below the rear side 46 of the top cover 20. The scent tube 18 includes a recess 56 that extends from the bottom end 54 to a position within the top portion 48 (as shown in FIG. 2). The recess 56, as shown, is generally cylindrical; however, the recess 56 in the bottom portion 51 has a slightly larger diameter to create a position for inserting the magnet 22 (discussed infra). The middle portion 50 of the scent tube 18 further includes at least one hole 58 and, more preferably, a series of holes 58 that extend from the recess 56 through the scent tube 18 at a position that, when the scent tube 18 is fully inserted into the top cover 20, the hole(s) 58 are positioned below the lower end 36 of the top cover 20. Although the hole(s) 58 can be positioned around the around periphery of the bottom end 54 of the scent tube 18, the hole(s) 58 are preferably positioned towards the front side of the scent tube 18. The hole(s) can 58 be sized and shaped in any manner desired by the designer. The scent tube 18 is preferably made of plastic or metal. Most preferably, the scent tube is made of brass.

The cylindrical magnet 22 has an outer diameter sized to slide into the bottom portion 51 of the scent tube and an inner diameter that is larger than the size of the scent stick (discussed infra). The magnet 22 is positioned in the bottom portion of the scent tube 18, as shown in FIG. 2 and is intended to maintain the replaceable scent stick insert in place during use.

Referring now to FIGS. 2, 3 and 5, the replaceable scent stick insert 14 includes a scent stick 30 that is affixed to a cartridge base 31. The scent stick 30 is sized to be inserted into the recess 56 in the scent tube 18 during use. The scent stick 30 is made of an absorbant material that has aromatic substance absorbed therein that, preferably, provides one or more aromatherapy benefits to the user. The absorbent material can be made of foam, or other sponge-like, material. Alternatively, the absorbent material can be made of ceramic, bamboo, cotton or Porex™.

The cartridge base 31 is generally a round cylinder with a recess 62 into which the scent stick 30 is affixed. The cartridge insert 31 is preferably made of a metal, such as steel, so that it will be held in place by the magnet 22 during use.

Substances that can be absorbed by the scent stick 30 to provide aromatherapy to the end user can include, but are not limited to essential oils, absolutes, carrier oils, herbal distillates (or hydrosols), and infusions. The substances can be applied by a manufacturer prior to packaging and sold to an end user for use with the apparatus 10, or a user may choose to purchase an unscented scent stick and apply the substance him- or herself

The bottom assembly 16 includes a magnet 28, a spacer 26, and a bottom insert 32 inserted into the bottom cover 24. The bottom cover 24 is generally cylindrical and extends between an upper end 64 and a lower end 66. The outer shape of the bottom assembly 16, in some embodiments, generally matches the outer shape of the top assembly 12; however, in other embodiments (not shown), they may differ. The bottom cover 24 includes an upper portion 68 that has an inner diameter that is smaller than the inner diameter of the lower portion 70. The difference in diameters creates a ledge, as shown in FIG. 2 designed to provide a stop when the magnet 28 is inserted into the bottom cover 24. The magnet 28 is utilized during use to retain bottom assembly 16 on the top assembly 12 during use. The magnet 28 can also include connection features 72 that mate with corresponding connection features 74 on the scent tube 18 that help keep the bottom assembly 16 attached to the top assembly 12 during. However, the size of the magnet 28 and/or the connection features 72 and corresponding connection features 74 are designed so that a reasonable pre-determined force applied by a user will separate the top and bottom assemblies 12, 16 so that the replaceable stick insert 14 can be easily replaced. A spacer 26 is inserted into the bottom cover 24 after the magnet 28. The spacer 26 can be made of any material, including plastics or metals. The inner diameter of the spacer 26 is sized to pass over cartridge base 31 when the top and bottom assemblies 12, 16 are put together. The upper end 64 of the bottom cover 24 is angled, preferably at an angle that is complimentary to the angle of the lower end 36 of the top cover 20. As is shown in FIGS. 2 and 3, the lower end 66 of the bottom cover 24 is generally at a 90 degree angle relative to the length of the cylindrical shape. The bottom insert 32 provides a cover for the bottom cover 24.

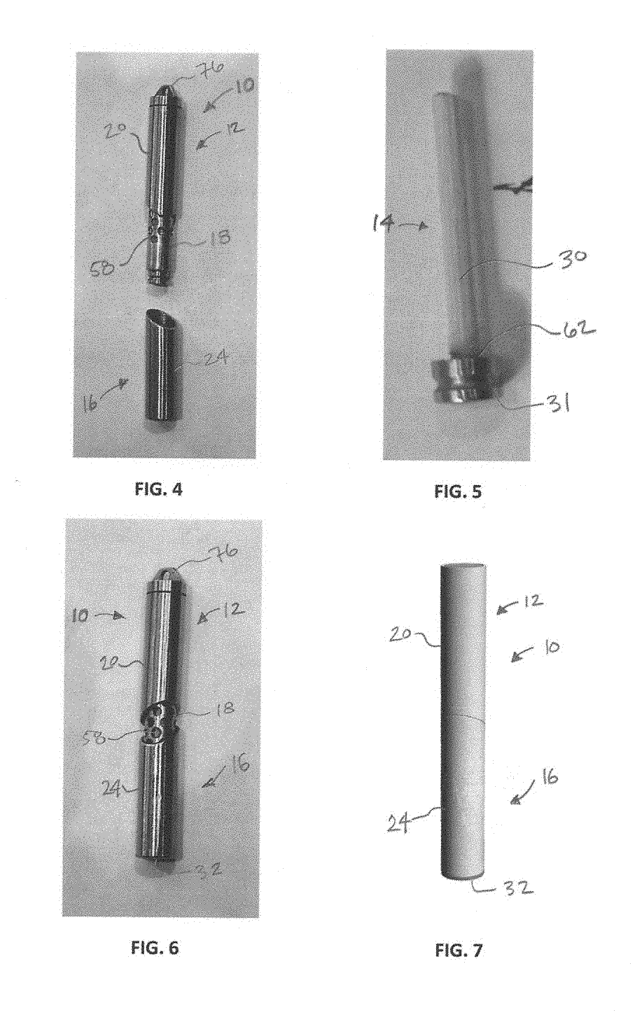

The top assembly, in some embodiments, can further include a hole 76 through which a necklace chain can be threaded, or can include a clasp or other connection mechanism known in the art. Examples are shown in FIGS. 1, 4 and 6. Preferably, the holes 76 or other clasp mechanism are positioned such that, when the assembly 10 is worn by a user, the holes 58 in the scent tube 18 face away from the user for maximum effectiveness.

During use, a user can purchase a replaceable stick insert 14 that is either unscented, or pre-loaded with a substance that is known to provide aromatherapy. If unscented, the end user can apply a substance of his or her own preference to the scent stick 30 to provide the desired aromatherapy benefit(s). The user then separates the top assembly 12 from the bottom assembly 16 and the replaceable stick insert is inserted into the recess 56 of the scent tube 18 until the metallic cartridge base 31 connects with the magnet 22 to hold the replaceable stick insert 14 in place. The bottom assembly 16 is then positioned onto the top assembly 12 and the replaceable stick insert 14 until the magnet 28 and/or connection features 72 of the retaining clip 28 mate with the complimentary connection features 74 on the scent tube 18.

Referring now to FIGS. 2, 6, and 7, the user can then determine, by rotating the bottom assembly 16 relative to the top assembly 12 how much of the hole(s) are covered. For example, when the user positions the angled lower end 36 of the top cover 20 and the complimentary angled upper end 64 of the bottom cover 24 such that the angled surfaces are mating (see FIG. 7), the substance of the scent stick will not emit from the assembly 10. Alternatively, if the user rotates the bottom assembly 16 by 180 degrees, the holes will be uncovered entirely, the substance will be emitted from the assembly during use.

While the present invention has been described in its preferred embodiments, it is to be understood that the invention is not limited thereto; but may be otherwise embodied. For example, while a necklace has been described, the apparatus 10 can include other types of fashionable jewelry, such as a ring, bracelet, broach or earrings. In addition, the article can be handheld by the user, as opposed to attached to a necklace chain/worn as a piece of fashionable jewelry. In addition, while magnets are conveniently used to hold the various pieces together during use, press fits and/or connection features known to those of skill in the art can be substituted without departing from the spirit and scope of the present invention.

I claim:

1. An apparatus for use as a fashionable jewelry, comprising:
a scent tube positioned in a top cover, the scent tube having a recess and at least one hole that extends from the recess to an outer surface of the scent tube;
a replaceable stick insert that is positioned within recess of the scent tube, the replaceable stick insert having a scent stick that includes a substance that provides one or more aromatherapy benefits;
a bottom cover that is rotatable relative to the scent tube from an open position to a closed position.

2. The apparatus of claim 1, wherein a cylindrical magnet is positioned within a recess in the scent tube; and
wherein the replaceable stick insert include a metallic base that connects to the cylindrical magnet during use.

3. The apparatus of claim 1, wherein the top cover includes a lower end and the bottom cover includes an upper end that are angled relative to the generally cylindrical such that when the user rotates the top cover relative to the bottom cover, the user may select how much or how little of the scent tube is exposed.

4. The apparatus of claim 1 wherein the top cover further includes a connection mechanism to attach the apparatus to a necklace.

5. A replaceable stick insert, comprising:
a scent stick that is made of a first material, the scent stick being generally cylindrical and defining a radius;
a base attached to one of the scent stick, the base being metallic and defining a second radius, the second radius being larger than the first radius;
wherein the scent stick is at least partially made of an absorbant material, and
wherein the absorbant contains at least one aromatic substance.

6. The replaceable stick insert of claim 5, wherein the scent stick is made entirely of absorbant material.

7. The replaceable stick insert of claim 5, wherein the scent stick is made at least partially of bamboo.

8. The replaceable stick insert of claim 5, wherein the scent stick is made at least partially of Porex™.

9. The replaceable stick insert of claim 5, wherein the scent stick is made at least partially of cotton.

10. The replaceable stick insert of claim 5, wherein the scent stick is made at least partially of ceramic material.

11. The replaceable stick insert of claim 5, wherein the scent stick is affixed to the base by press fitting the scent stick to the base.

12. The replaceable stick insert of claim 5, wherein the aromatic substance provides at least one aromatherapy benefit.

13. The replaceable stick insert of claim 5, wherein the aromatic substance is selected from one or more of the following: essential oils, absolutes, carrier oils, herbal distillates (or hydrosols), and infusions.
